(19) United States
(12) Patent Application Publication
(10) Pub. No.: US 2026/0218300 A1
(43) Pub. Date: Jul. 30, 2026

(54) BIOMARKER FOR DIAGNOSIS OF ANXIETY DISORDERS AND USE THEREOF

(71) Applicants: CHA UNIVERSITY INDUSTRY-ACADEMIC COOPERATION FOUNDATION, Pocheon-si, Gyeonggi-do (KR); SUNGKWANG MEDICAL FOUNDATION, Seoul (KR)

(72) Inventors: Min Soo KWON, Seoul (KR); Sang Hyek LEE, Seongnam-si (KR); Minji BANG, Seongnam-si (KR); Min Jung YOU, Anyang-si (KR); Chan RIM, Suwon-si (KR)

(21) Appl. No.: 19/147,129

(22) PCT Filed: Jan. 11, 2024
(86) PCT No.: PCT/KR2024/000534

§ 371 Date: Jul. 10, 2025

(30) Foreign Application Priority Data
Jan. 11, 2023 (KR) 10-2023-0004150

(51) Int. Cl. C12Q 1/6883; C12Q 1/48

(52) U.S. Cl. CPC C12Q 1/6883; C12Q 1/48; C12Q 2600/112; C12Q 2600/158; C12Y 203/01009; G01N 2800/301; G01N 2800/60

(57) ABSTRACT

Provided are a composition and a diagnostic kit for diagnosing anxiety disorders, including a preparation for measuring the expression level of mRNA of an Acetyl-CoA acetyltransferase 2 (ACAT2) gene or a protein encoded by the gene for diagnosing anxiety disorders, and a diagnostic method including measuring the expression level of mRNA of the ACAT2 gene or a protein encoded by the gene.

### TECHNICAL FIELD

The present application claims priority to Korean Patent Application No. 10-2023-0004150, filed on Jan. 11, 2023, which is hereby incorporated by reference in its entirety.

Provided are a composition for diagnosing anxiety disorders, a kit, and a method of diagnosing anxiety disorders using the same.

### BACKGROUND ART

Microglia are innate immune cells that reside in the brain parenchyma and are involved in maintaining homeostasis within the brain. To maintain homeostasis, microglia remove apoptotic cells, necrotic cells resulting from injury, and cellular debris or toxic proteins such as amyloid-β through phagocytosis. Additionally, they secrete nutrients to help nerve cells survive and protect nerve cells from invasion by foreign substances through immune activity.

Anxiety disorder is a general term for mental illness that causes difficulties in daily life due to various forms of abnormal and pathological anxiety and fear. Anxiety and fear are normal emotional responses that serve as warning signals of impending danger, but when excessive, they may make it more difficult to cope with situations appropriately and may cause mental distress and physical symptoms. Anxiety may be diagnosed as an anxiety disorder when the sympathetic nervous system is stimulated by anxiety, causing physical symptoms such as headaches, increased heart rate, increased breathing rate, and gastrointestinal symptoms, which cause discomfort, and when anxiety, worry, or physical symptoms cause difficulties in daily activities such as work, interpersonal relationships, and schoolwork. Specifically, anxiety disorders include phobic disorder, generalized anxiety disorder, obsessive compulsive disorder, post-traumatic stress disorder, panic disorder, agoraphobia, separation anxiety disorder, selective mutism, and the like. A representative anxiety disorder, phobic disorder, refers to a disorder in which an individual experiences irrational fear of a specific object or situation and persistently avoids the object or situation because of this, and there are many types depending on the object, such as social phobia, acrophobia, claustrophobia, expectation anxiety, and the like. Generalized anxiety disorder refers to a case where one feels widespread and persistent anxiety in everyday situations, and obsessive-compulsive disorder refers to a case where one repeatedly has certain thoughts or behaviors regardless of one's will, and a certain degree of obsessive thoughts or behaviors may be present even in normal times. Therefore, if obsessive-compulsive symptoms interfere with one's daily life or cause mental and physical distress, the condition is diagnosed as obsessive-compulsive disorder, which usually exhibits obsessive personality traits, such as accuracy, perfectionism, and principled behavior. Post-traumatic stress disorder is a psychological reaction that occurs after experiencing mental trauma, such as experiencing or witnessing life-threatening extreme stress, in other words, a shocking and frightening event, and even after the trauma has passed, the person continues to recall the traumatic memory, avoids activities or places that remind them of the trauma, may lose control over what will happen in the future, feel fear, and exhibits symptoms such as autonomic nervous system symptoms. Additionally, panic disorder is an anxiety disorder in which severe anxiety attacks and various accompanying physical symptoms occur suddenly without warning.

However, since there is still a lack of understanding of the neural circuits that regulate anxiety symptoms, their operating mechanisms, and the factors that regulate them, further research on the neural circuits involved in anxiety symptoms is necessary, and based on this, it is necessary to develop a diagnostic method capable of effectively diagnosing anxiety disorders.

### DISCLOSURE OF INVENTION

### Technical Problem

One aspect provides a composition for diagnosing anxiety disorder, or a diagnostic kit, including a preparation for measuring the expression level of mRNA of the Acyl-CoA cholesterol acyltransferase-2 (ACAT2) gene or a protein encoded by the gene.

Another aspect is a method of providing information necessary for diagnosing an anxiety disorder, including: (a) measuring the expression level of the mRNA of the ACAT2 gene or the protein encoded by the gene from an isolated biological sample; (b) comparing the expression level of the mRNA of the ACAT2 gene or the protein encoded by the gene measured in (a) with the expression level of the mRNA of the ACAT2 gene or the protein encoded by the gene of a normal control group sample; and (c) diagnosing an anxiety disorder when the expression level of the mRNA of the ACAT2 gene or the protein encoded by the gene of the biological sample is higher than the expression level of the mRNA of the ACAT2 gene or the protein encoded by the gene of a normal control group sample.

### Solution to Problem

One aspect relates to a composition for diagnosing anxiety disorder, including a preparation for measuring the expression level of mRNA of the Acyl-CoA cholesterol acyltransferase-2 (ACAT2) gene or a protein encoded by the gene.

As used herein, the term “Acyl-CoA cholesterol acyltransferase-2 (ACAT2)” refers to an acyltransferase that forms cholesteryl ester from cholesterol and a gene encoding the same, and may be used interchangeably with Sterol O-acyltransferase (SOAT). The term “ACAT2” may refer to the ACAT2 protein or the gene encoding it. For example, the substrates of the ACAT2 protein may be acyl-CoA and cholesterol, and the products may be CoA and cholesteryl ester. Therefore, the increase in ACAT2 may be involved in reducing cellular cholesterol levels and increasing the levels of cholesteryl esters, which are also involved in lipid droplet formation.

As used herein, the term “anxiety disorder” refers to a mental illness in which anxiety appears at an abnormal level different from normal, and for example, the anxiety disorder may be any one disease selected from the group consisting of phobic disorder, generalized anxiety disorder, obsessive compulsive disorder, post-traumatic stress disorder, panic disorder, agoraphobia, separation anxiety disorder, and selective mutism, but is not limited to the above diseases. For example, the anxiety disorder may be a panic disorder.

As used herein, the term “diagnosis” broadly refers to determining the actual condition of a patient's illness in all aspects. The contents of the determination are the name of disease, cause of disease, type of disease, severity, detailed condition of the disease, and presence or absence of complications, etc. In the present disclosure, diagnosis is to determine whether an anxiety disorder has occurred and the level of progression, etc.

As used herein, the term “expression level” refers to the amount of mRNA of the ACAT2 gene and/or protein encoded by the ACAT2 gene derived from a limited number of cells or a limited tissue portion, for example, the amount of ACAT2 mRNA and/or ACAT2 protein obtainable from standard nucleic acid (for example, RNA) or protein extraction procedures. Suitable extraction methods are known to experts in the field.

The method of measuring the mRNA expression level of the above gene may be a method of measuring the level of mRNA transcribed from the target gene to confirm whether the target gene included in the sample is expressed, and includes, for example, without limitation, methods such as RT-PCR, competitive RT-PCR, real-time RT-PCR, RNase protection assay (RPA), Northern blotting, and DNA chip analysis, etc.

The method of measuring the expression level or activity level of the above protein refers to a method of measuring the expression or activity level of a target protein included in a sample, and includes, for example, without limitation, methods such as western blotting, enzyme linked immunosorbent assay (ELISA), radioimmunoassay (RIA), radioimmunodiffusion, Ouchterlony immunodiffusion, rocket immunoelectrophoresis, tissue immunostaining, immunoprecipitation assay, complement fixation assay, FACS, and protein chip assay, etc.

In an embodiment, the measurement of the expression level of the mRNA or protein may be measured in microglial cells of the subject. For example, the microglia may be induced from peripheral blood mononuclear cells (PBMC) isolated from the subject, and may be prepared by treating peripheral blood mononuclear cells isolated from the subject's blood with recombinant human granulocyte-macrophage colony-stimulating factor (GM-CSF) and recombinant human interleukin-34 (IL-34). Microglia induced from the above peripheral blood mononuclear cells may be referred to as induced microglia cells.

In an embodiment, the composition may further include an agent for measuring the expression level of mRNA of any one gene selected from the group consisting of T-cell death-associated gene-8 (TDAG8), Triggering receptor expressed on myeloid cells 2 (TREM2), 7-dehydrocholesterol reductase (DHCR7), and progranulin (GRN) or a protein encoded by the gene.

Another aspect of the present disclosure relates to a kit for diagnosing anxiety disorder, including a preparation for measuring the expression level of mRNA of the ACAT2 gene or a protein encoded by the gene.

Among the terms or elements mentioned in the description of the above anxiety disorder diagnostic composition, those that are identical to those already mentioned are as described above.

The diagnostic kit of the present disclosure includes one or more different component compositions, solutions or devices suitable for the analysis method. For example, the kit of the present disclosure may be a kit including genomic DNA derived from a sample to be analyzed, a primer set specific for the marker gene of the present disclosure, an appropriate amount of DNA polymerase, a dNTP mixture, a PCR buffer solution, and water to perform PCR. The above PCR buffer solution may contain KCl, Tris-HCl and MgCl₂. In addition, components necessary for performing electrophoresis to confirm whether a PCR product has been amplified may be additionally included in the kit of the present disclosure.

Additionally, the kit of the present disclosure may be a kit including essential elements required for performing RT-PCR. In addition to each primer pair specific for a marker gene, an RT-PCR kit may include a test tube or other appropriate container, reaction buffer solution, deoxynucleotides (dNTPs), enzymes such as Taq polymerase and reverse transcriptase, DNase and RNase inhibitors, DEPC-water, sterile water, etc. It may also include primer pairs specific for the genes used as a quantitative control group.

Additionally, the kit of the present disclosure may be a kit including essential elements required for performing a DNA chip. The DNA chip kit includes a substrate to which a cDNA corresponding to a gene or a fragment thereof is attached as a probe, and the substrate may include a cDNA corresponding to a quantitative structural gene or a fragment thereof. Additionally, the kit of the present disclosure may be in the form of a microarray having a substrate on which the marker gene of the present disclosure is immobilized.

Additionally, the kit of the present disclosure may be a kit characterized by including essential elements necessary for performing ELISA. ELISA kits include specific antibodies against a marker protein and a preparation for measuring the level of said protein. The above ELISA kit may include reagents capable of detecting antibodies that have formed an “antigen-antibody complex,” for example a labeled secondary antibody, chromophores, enzymes, and substrates thereof. Additionally, it may include an antibody specific for the quantitative control group protein.

Another aspect of the present disclosure relates to a method of providing information necessary for diagnosing an anxiety disorder, including: (a) measuring the expression level of the mRNA of the ACAT2 gene or the protein encoded by the gene from an isolated biological sample; (b) comparing the expression level of the mRNA of the ACAT2 gene or the protein encoded by the gene measured in (a) with the expression level of the mRNA of the ACAT2 gene or the protein encoded by the gene of a normal control group sample; and (c) diagnosing an anxiety disorder when the expression level of the mRNA of the ACAT2 gene or the protein encoded by the gene of the biological sample is higher than the expression level of the mRNA of the ACAT2 gene or the protein encoded by the gene of a normal control group sample.

Among the terms or elements mentioned in the description of the above anxiety disorder diagnostic kit, those that are identical to those already mentioned are as described above.

As used herein, the term “method of providing information necessary for diagnosing anxiety disorder” is a preliminary step for diagnosis, providing objective basic information necessary for diagnosing anxiety disorder, and excluding a doctor's clinical judgment or opinion.

As used herein, the term “normal control group” refers to a subject who does not have an anxiety disorder.

The above separated biological samples may include, but are not limited to, tissues, cells, whole blood, blood, saliva, sputum, cerebrospinal fluid, and urine. For example, the biological sample may be blood or plasma. For example, the biological sample may be peripheral blood mononuclear cells or microglial cells, and the microglial cells may be induced from peripheral blood mononuclear cells isolated from the subject, for example, prepared by treating peripheral blood mononuclear cells isolated from the subject with recombinant human granulocyte-macrophage colony-stimulating factor (GM-CSF) and recombinant human interleukin-34 (IL-34). For example, the biological sample may be an induced microglia cell derived from peripheral blood mononuclear cells.

### Advantageous Effects of Invention

According to a diagnostic composition, a diagnostic kit, or a diagnostic method according on an aspect, an anxiety disorder may be diagnosed with high accuracy, and thus may be usefully utilized in the diagnosis of an anxiety disorder.

### BRIEF DESCRIPTION OF DRAWINGS

FIG. 1 is a diagram illustrating a differentiation process from peripheral blood mononuclear cells (PBMCs) into induced microglia cells (iMGs) over time.

FIG. 2 shows results confirming the mRNA expression levels of microglia-specific genes MafB, Csf1r, Gpr34, Hexb, and C1qa in PBMCs and iMGs of each panic disorder patient by qRT-PCR. The Relative Quantity (RQ) value for each gene represents a percentage of the expression level in iMGs compared to PBMCs of panic disorder patients.

FIG. 3 shows confocal microscope fluorescence images and a graph showing results of mean fluorescence intensity (MFI) quantification, illustrating the expression level of TDAG8 in iMGs of a normal control group and iMGs of panic disorder patients confirmed through immunofluorescence staining.

FIG. 4 is graphs illustrating mRNA expression levels of TDAG8 in PBMCs and iMGs of a normal control group and panic disorder patients.

FIG. 5A is a diagram illustrating a correlation between the mRNA expression level of TDAG8 in peripheral blood mononuclear cells and panic disorder clinical scores through Spearman correlation analysis.

FIG. 5B is a diagram illustrating a correlation between the mRNA expression level of TDAG8 in induced microglia cells and panic disorder clinical scores through Spearman correlation analysis.

FIG. 6 is confocal microscope fluorescence images and a graph, illustrating phagocytosis of microglia in iMGs of normal control groups and panic disorder patients.

FIG. 7 is graphs illustrating mRNA expression levels of TREM2 in PBMCs and iMGs of a normal control group and PD patients.

FIG. 8 is a diagram illustrating a correlation between the mRNA expression level of TREM2 and panic disorder clinical scores through Spearman correlation analysis.

FIG. 9 is a graph illustrating mRNA expression levels of each gene in iMGs of a normal control group and PD patients.

FIG. 10 is graphs illustrating mRNA expression levels of ACAT2 in PBMCs and iMGs of a normal control group and PD patients.

FIG. 11 is a graph illustrating mRNA expression levels of DHCR7 in PBMCs and iMGs of a normal control group and PD patients.

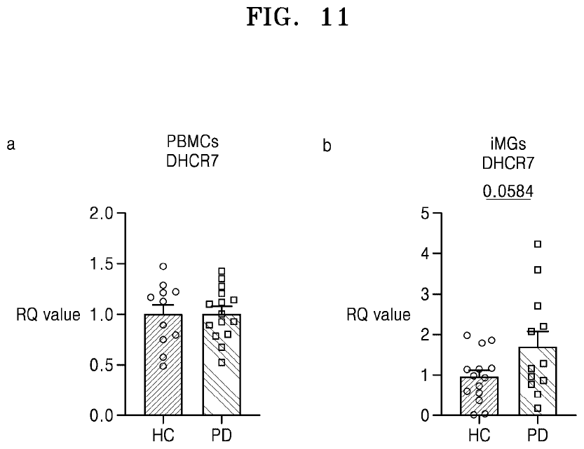

FIG. 12 is a diagram illustrating a correlation between the mRNA expression level of ACAT2 and panic disorder clinical scores through Spearman correlation analysis.

FIG. 13 is a diagram illustrating a correlation between the mRNA expression level of DHCR7 and panic disorder clinical scores through Spearman correlation analysis.

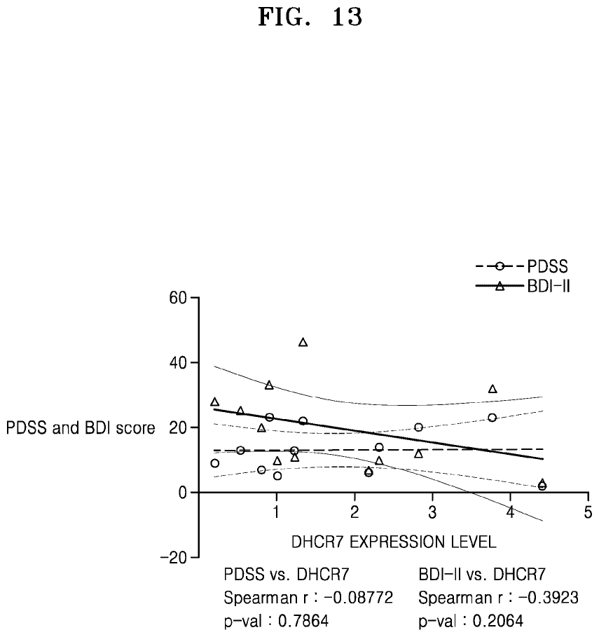

FIG. 14 is a confocal microscope fluorescence images illustrating the formation of lipid droplets in iMGs of a normal control group and iMGs of a panic disorder patient through immunochemical staining.

FIG. 15 is graphs illustrating mRNA expression levels of GRN in iMGs and PBMCs of a normal control group and panic disorder patients.

### BEST MODE FOR CARRYING OUT THE INVENTION

The inventors of the present disclosure isolated peripheral blood mononuclear cells (PBMCs) from blood samples of panic disorder patients, established induced microglia cells (iMG) therefrom, and performed mRNA analysis of these cells. Through this, specific genes with significant expression levels within iMG were selected, and a correlation between the expression levels of the specific genes and panic disorder symptoms was confirmed, and the present disclosure was completed based on this.

iMGs are not simply assumed to be identical to yolk sac macrophage-derived microglia, as mRNA profiling revealed that iMGs are closely clustered with human primary microglia and pluripotent stem cell-derived microglia (Banerjee A, Lu Y, Do K, Mize T, Wu X, Chen X, et al. Validation of Induced microglia cells (IMG Cells) for Future Studies of Brain Diseases. Front Cell Neurosci 2021; 15:629279). Accordingly, the iMG of the inventors of the present disclosure may serve as a potential cell model system to investigate human microglial pathology in living patients with microglial-associated brain disorders.

The present study was reviewed and approved by the Institutional Review Board of Bundang Cha Hospital in accordance with the Declaration of Helsinki and the principles of Good Clinical Practice (IRB No. 2019-05-030).

### Example 1. Selection of Participants

Panic disorder patients were recruited at the Department of Psychiatry, CHA Bundang Medical Center (Seongnam, Republic of Korea) from August 2019 to May 2021. The diagnosis of panic disorder was made by an experienced psychiatrist based on the Diagnostic and Statistical Manual of Mental Disorders, Fifth Edition (DSM-5) criteria using the Structured Clinical Interview for DSM-5 Disorders. Only participants diagnosed with primary panic disorder were included. Participants were excluded if they: (1) had a history of major mental disorders including psychotic disorders, substance use disorders, bipolar disorder, or major depressive disorder; (2) had a history of neurological disorders, traumatic brain injury, or intellectual disability (IQ<70); (3) had clinically significant medical illnesses; or (4) were pregnant. Normal control groups were recruited from the local population through online and print advertisements. Individual interviews by experienced psychiatrists confirmed that there was no history of mental illness in the individual or their first-degree relatives. The exclusion criteria for the above group were applied identically to those used for the panic disorder patients. All participants were of Korean ethnicity. Finally, 17 panic disorder patients and 16 normal control subjects were included in this study, and the information thereon is presented in Table 1 below.

TABLE 1

| | | | PBMC | Blood | RNA Integrity Number |
|---|---|---|---|---|---|
| Sample | Sex | Age | yield | volume | (Bioanalyzer) (1~10) |
| PD1 | F | 56 | 50,000,000 | 50 ml | 8.9 |
| PD2 | M | 50 | 30,000,000 | 50 ml | — |
| PD3 | F | 41 | 136,000,000 | 60 ml | 8.5 |
| PD4 | F | 30 | 32,000,000 | 20 ml | 8.5 |
| PD5 | M | 30 | 34,000,000 | 30 ml | — |
| PD6 | M | 36 | 79,000,000 | 30 ml | 8.7 |
| PD7 | F | 26 | 48,000,000 | 30 ml | 9.5 |
| PD8 | M | 42 | 33,000,000 | 30 ml | 9.0 |
| PD9 | M | 61 | 22,500,000 | 30 ml | 9.6 |
| PD10 | M | 16 | 45,700,000 | 30 ml | 8.9 |
| PD11 | F | 50 | 28,200,000 | 30 ml | 9.4 |
| PD12 | F | 40 | 54,600,000 | 30 ml | 9.0 |
| PD13 | M | 42 | 80,000,000 | 40 ml | — |
| PD14 | F | 22 | 70,000,000 | 30 ml | 7.5 |
| PD15 | F | 24 | 39,000,000 | 30 ml | 9.1 |
| PD16 | F | 20 | 40,000,000 | 30 ml | 9.1 |
| PD17 | M | 21 | 45,000,000 | 30 ml | 9.0 |
| HC1 | M | 42 | 90,000,000 | 50 ml | 9.5 |
| HC2 | M | 27 | 31,000,000 | 30 ml | 8 |
| HC3 | F | 30 | 38,000,000 | 40 ml | 7.6 |
| HC4 | M | 35 | 28,700,000 | 30 ml | — |
| HC5 | F | 40 | 32,500,000 | 30 ml | — |
| HC6 | F | 34 | 23,000,000 | 30 ml | — |
| HC7 | F | 38 | 60,000,000 | 30 ml | 9.0 |
| HC8 | M | 26 | 36,000,000 | 30 ml | 8.6 |
| HC9 | F | 28 | 48,000,000 | 30 ml | 8.3 |
| HC10 | F | 23 | 86,700,000 | 30 ml | 7.9 |
| HC11 | M | 40 | 50,000,000 | 30 ml | 7.3 |
| HC12 | M | 49 | 59,000,000 | 30 ml | 9.6 |
| HC13 | F | 46 | 34,000,000 | 20 ml | 9.5 |
| HC14 | F | 62 | 27,000,000 | 30 ml | — |
| HC15 | F | 41 | 70,000,000 | 30 ml | — |
| HC16 | F | 43 | 52,000,000 | 30 ml | — |

### Example 2-1. Establishment of Induced Microglia Cells

Peripheral blood (~30 mL) was acquired from normal control groups and panic disorder patients using heparinized tubes. PBMCs were isolated by density gradient centrifugation using Lymphoprep™ (STEMCELL technologies, Catalog #07851/07861) and resuspended in Roswell Park Memorial Institute (RPMI) 1640 buffer (Gibco, Waltham, MA, USA) containing 10% FBS (Gibco) and 1% penicillin/streptomycin (Gibco). The above separated PBMCs were seeded at a density of 5×10⁵ cells/ml overnight in an incubator with 5% CO₂ at 37° C. The remaining PBMCs were used for qPCR analysis. After carefully aspirating the above medium, adherent cells (monocytes) were cultured for 21 days in RPMI 1640 Glutamax (Gibco) supplemented with recombinant human granulocyte-macrophage colony-stimulating factor (GM-CSF; 10 ng/ml; R&D Systems, Minneapolis, MN) and recombinant human interleukin-34 (IL-34; 100 ng/ml; R&D Systems) to establish induced microglia cells (iMG) based on a previously published protocol.

### Example 2-2. Confirmation of Differentiation of Induced Microglia Cells (IMG)

Since individual induced microglia cells (IMG) showed different cell yields despite high expression of microglia signature genes, the extent of molecular analysis was determined by the yield and quality control (QC) of individual induced microglia cells obtained (Table 1).

### (1) Confirmation of Morphological Changes

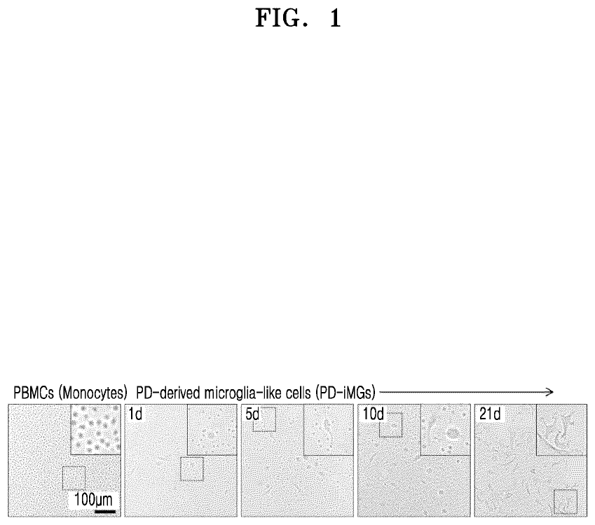

The results of induced microglia cells established through the above Example 2-1 are as shown in FIG. 1. The number of days shown in FIG. 1 refers to the number of days after the differentiation factor was added. After the above treatment, it was confirmed that the small spherical shapes expanded and changed into branched shapes over time (FIG. 1), confirming differentiation into induced microglia cells.

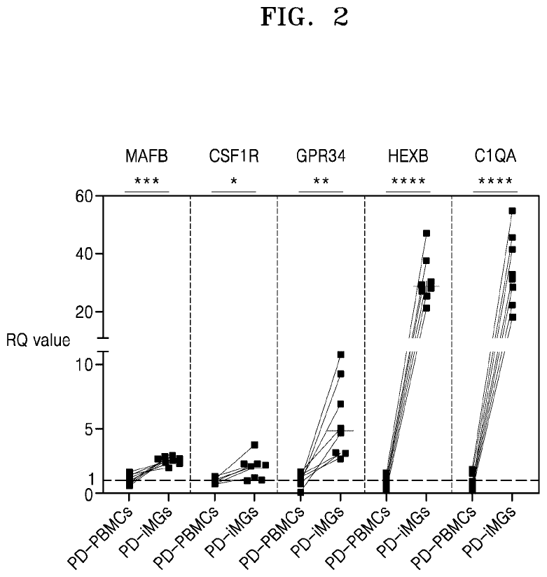

(2) Confirmation of Increased Expression Levels of Microglia Signature Genes mRNA expression levels of microglial signature genes (MAFB, CSF1R, C1QA, GPR34, HEXB, and GAPDH (PPH00150F)) were analyzed by quantitative PCR compared to PBMCs. As a result, it was confirmed that the expression of microglia-specific genes, including Mafb, Csf1r, Gpr34, Hexb, and C1qa, was significantly increased in iMG of panic disorder patients compared to PBMC (FIG. 2), confirming differentiation into induced microglia cells. The RQ values in FIG. 2 refers to the percentage of each gene expression level in induced microglia cells relative to monocytes.

### Example 3-1. Confirmation of Correlation Between mRNA Expression Level of TDAG8 Gene in Induced Microglia Cells of Panic Disorder Patients and Panic Disorder Symptoms

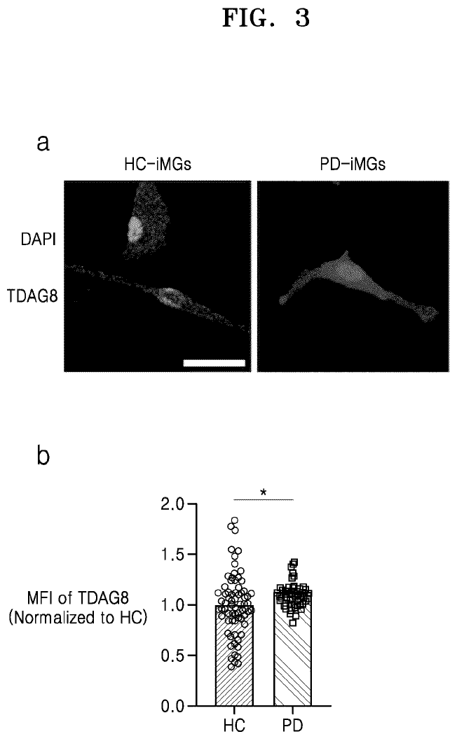

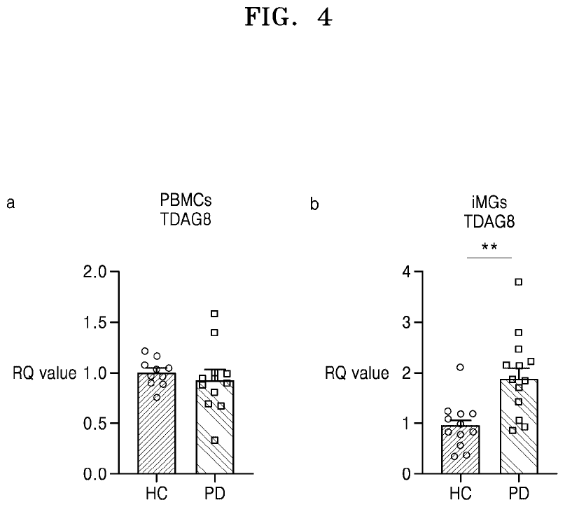

The mRNA expression levels of a TDAG8 gene were compared in PBMCs and induced microglia cells from panic disorder patients and normal control groups, respectively. In the case of PBMC, no difference in the expression level of TDAG8 mRNA was observed between panic disorder patients and normal control groups. In contrast, in the case of induced microglia cells, it was confirmed that TDAG8 expression levels were higher in panic disorder patients than in normal control groups using immunofluorescence (FIG. 3) and quantitative PCR (FIG. 4).

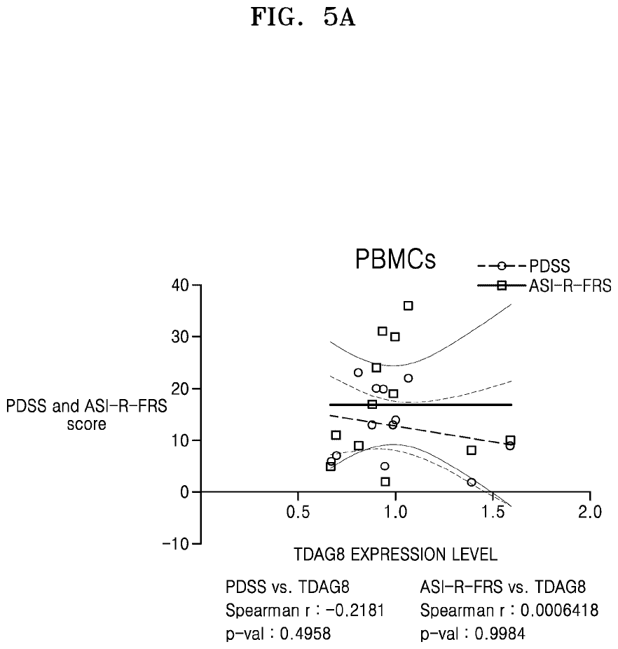

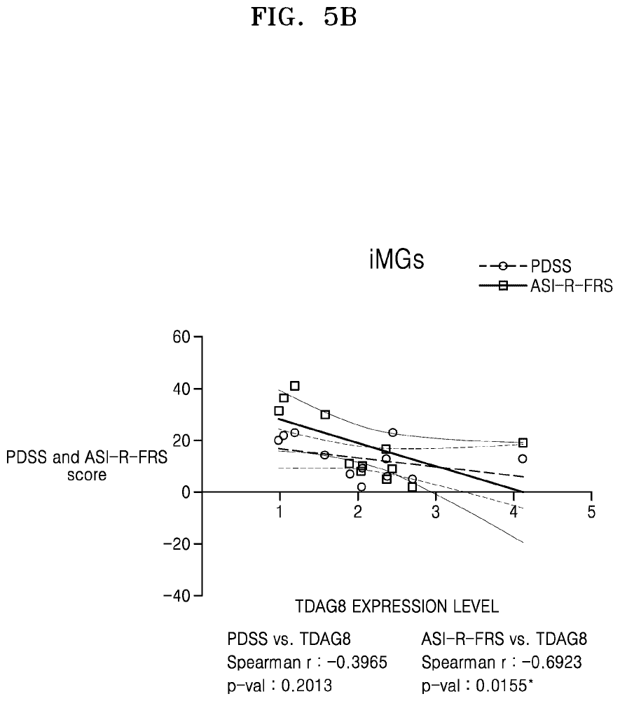

Additionally, using the Anxiety Sensitivity Index-Revised (ASI-R), it was confirmed that increased TDAG8 expression in induced microglia cells of panic disorder patients was significantly inversely correlated with fear of respiratory symptoms (FIG. 5). In contrast, no correlation was found between the expression level of TDAG 8 and fear of respiratory symptoms in the case of PBMCs of panic disorder patients.

Through this, it was confirmed that microglia of panic disorder patients are excellent proxy cells that reflect the brain physiology of panic disorder patients better than PBMC. This may be related to the fact that microglia play a role in the survival and protection of nerve cells, and that anxiety disorders such as panic disorder may be affected by high stress, abnormal neural activity, and neuroinflammatory responses, etc.

### Example 3-2. Confirmation of Correlation Between Decreased Phagocytosis and Increased mRNA Expression Level of TREM2 Gene in Induced Microglia Cells of Panic Disorder Patients and Panic Disorder Symptoms

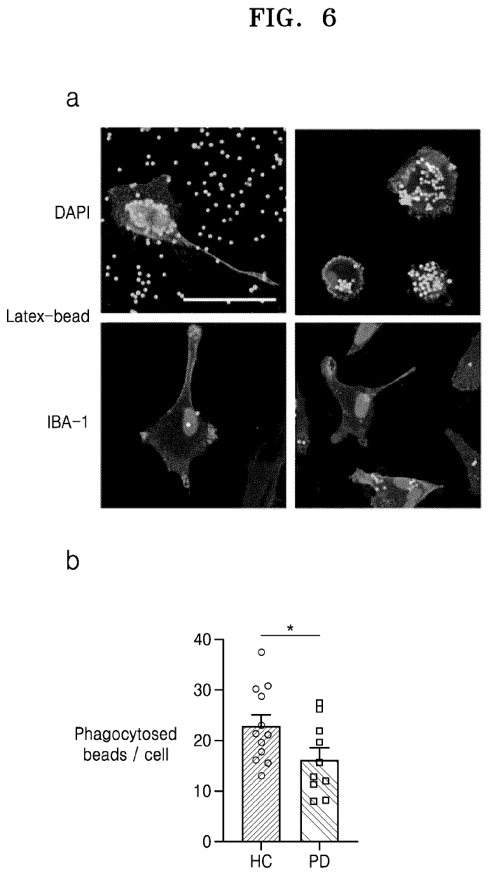

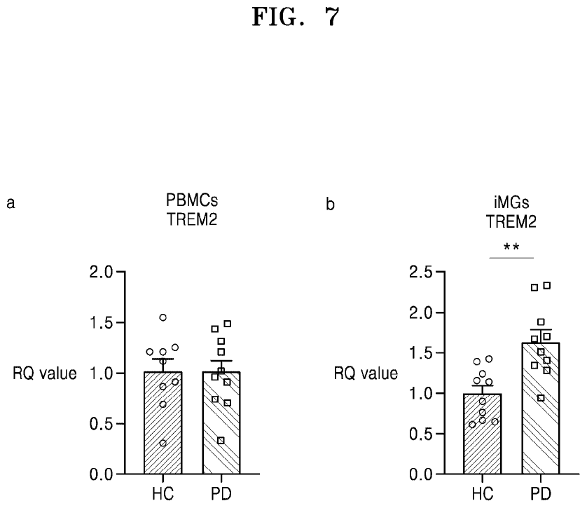

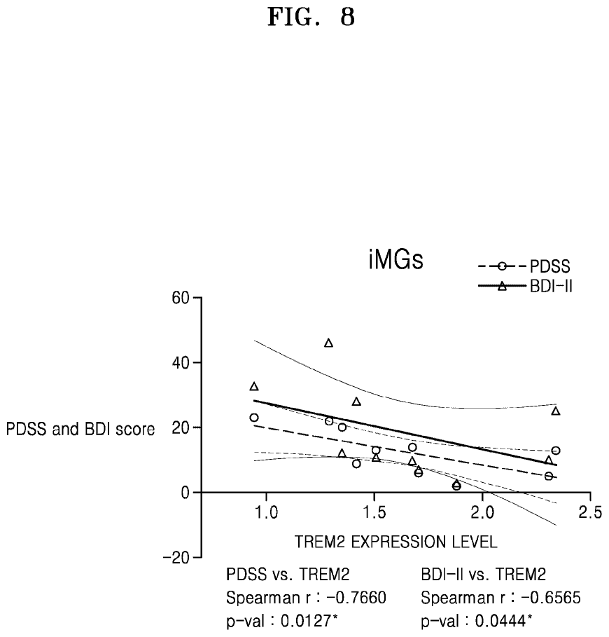

To evaluate the phagocytic function, a representative function of microglia, a phagocytosis assay was performed using latex beads. It was confirmed that induced microglia cells from panic disorder patients captured fewer latex beads than normal control groups (FIG. 6). Based on this, considering that TREM2 plays a central role in the phagocytic function of microglia, the mRNA expression level of the TREM2 gene was measured. As a result, in the case of PBMC, there was no significant difference in the degree of expression between the normal control group and panic disorder patients. In contrast, in the case of induced microglia cells, the expression level of the TREM2 gene mRNA in panic disorder patients was confirmed to be significantly higher than that in the normal control group (FIG. 7). Additionally, it was confirmed that increased TREM2 expression in induced microglia cells of panic disorder patients was negatively correlated with panic symptoms and comorbid depression (FIG. 8).

Through this, it was confirmed that microglia of panic disorder patients are excellent proxy cells that reflect the brain physiology of panic disorder patients better than PBMC.

### Example 3-3. Confirmation of Correlation Between Lipid Droplet Accumulation and Increased mRNA Expression Level of GRN Gene in Induced Microglia Cells of Panic Disorder Patients and Panic Disorder Symptoms

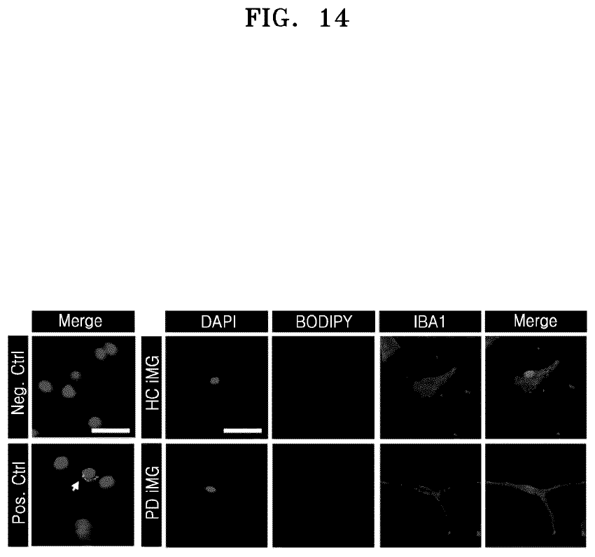

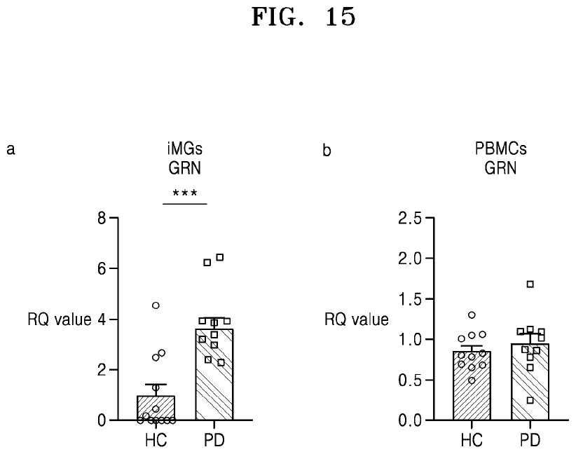

BODIPY staining was performed on induced microglia cells to analyze the accumulation of cholesterol and lipid metabolites in induced microglia cells. As shown in FIG. 14, it was confirmed that lipid droplet accumulation did not occur in induced microglia cells of panic disorder patients. Based on this, the mRNA expression level of the GRN (Granulin precursor) gene, an inhibitor associated with lipid droplet accumulation in induced microglia cells of panic disorder patients, was confirmed through quantitative PCR. In the case of PBMC, GRN expression levels did not show significant differences between panic disorder patients and normal control groups. In contrast, in the case of induced microglia cells, it was confirmed that the expression level of GRN was significantly increased compared to the induced microglia cells of the normal control group of panic disorder patients (FIG. 15). Additionally, GRN expression was confirmed to be negatively correlated with the severity of trait symptoms including harm avoidance (HA) and neuroticism (HA: Spearman r=−0.617, p-value=0.025 and neuroticism: Spearman r=−0.613, p-value=0.026).

Through this, it was confirmed that microglia of panic disorder patients are excellent proxy cells that reflect the brain physiology of panic disorder patients better than PBMC.

### Example 4. Transcriptome Analysis of Induced Microglia Cells of Panic Disorder Patients

Comparative transcriptome analysis was performed to analyze the molecular differences in microglia between panic disorder patients and normal control groups. In the induced microglia cells of panic disorder patients, 387 differentially expressed genes (DEGs) were confirmed to be differentially expressed with a fold change >1.5 and P-value <0.05, of which 199 were down-regulated and 188 were up-regulated.

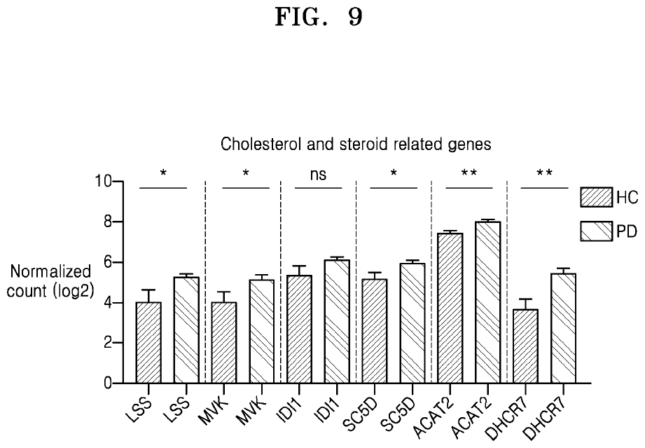

Additionally, hierarchical clustering analysis confirmed that induced microglia cells from panic disorder patients clustered together and were distinct from induced microglia cells from normal control groups. A series of biological processes that are altered and affected by the differentially expressed genes (DEGs) were analyzed through gene set enrichment analysis (GSEA). Using three databases (Reactome, KEGG, and WikiPathways (WP)), the top 10 altered biological processes are listed in Table 2, and six of them were confirmed to be related to cholesterol biosynthesis and steroid metabolism (Rank 3,4,5,6,8,10). Among these, the top four gene sets (Rank 3, 4, 5, 6) were selected and the core genes of each set were analyzed. Based on these results, the top 10 genes in each pathway were listed, and six genes were selected, including common Lss, Mvk, Idi1, Sc5d, Acat2, and Dhcr7. The Normalized Count (log 2) of the above six genes was compared (FIG. 9). Among these, the expression of genes except Idi1 was significantly up-regulated in induced microglia cells of panic disorder patients, and among these, Acat2 and Dhcr7 showed strong statistical significance. Through this, the Acat2 and Dhcr7 genes, which characteristically represent the properties of microglia in panic disorder patients, were discovered.

TABLE 2

| | | | Nominal |
|---|---|---|---|
| Rank | Database | Gene Set | p-value |
| 1 | Reactome | Amino Acids Regulate MTORC1 | <0.0001 |
| 2 | Reactome | Nuclear Envelope Reassembly | <0.0001 |
| 3 | Reactome | Cholesterol Biosynthesis | <0.0001 |
| 4 | Kegg | Steroid Biosynthesis | =0.003 |
| 5 | WP | Cholesterol Biosynthesis Pathway | =0.003 |
| 6 | Reactome | Metabolism of Steroids | <0.0001 |
| 7 | Reactome | Degradation of Axin | =0.001 |
| 8 | WP | Cholesterol metabolism with Bloch and | =0.002 |
| | | Kandutschrussel pathways | |
| 9 | WP | Methionine de novo and salvage pathway | =0.003 |
| 10 | Reactome | Regulation of cholesterol biosynthesis | =0.001 |
| | | by Srebp and Srebf | |

### Example 5. Screening of Biomarkers of Panic Disorder Patients

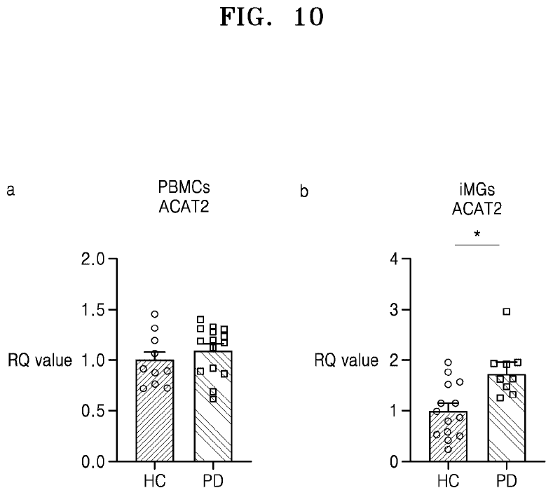

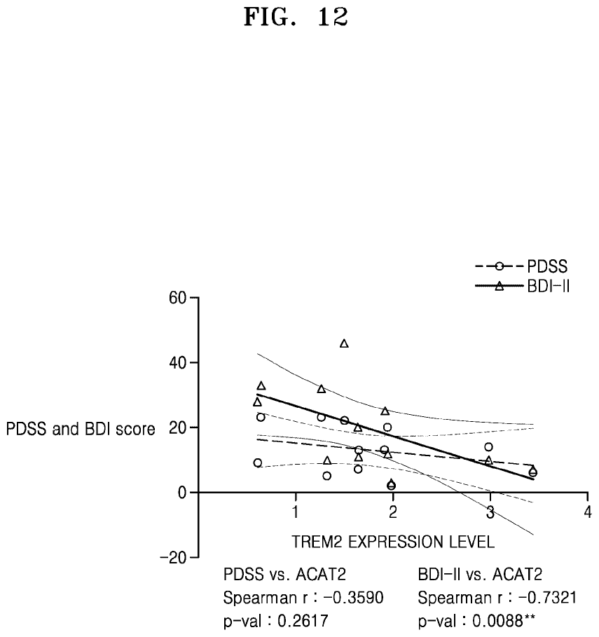

Based on the above transcriptome analysis, the expression levels of mRNA of the selected Acat2 and Dhcr7 genes were analyzed for microglia and PBMCs from normal control groups and panic disorder patients, respectively. In the case of PBMC, no significant difference was observed in the expression of the above genes between normal control groups and panic disorder patients. In contrast, in the case of induced microglia cells, it was confirmed that Acat2 expression was significantly increased in panic disorder patients compared to normal control groups (FIG. 10). Furthermore, it was confirmed that Acat2 expression in induced microglia cells of panic disorder patients was inversely correlated with depression severity and anxiety sensitivity to publicly observable anxiety reactions (FIG. 12). Through this, the gene Acat2, whose expression significantly increased in microglia of panic disorder patients compared to normal control groups, was selected as a biomarker for panic disorder patients.

### Materials and Methods

Quantitative Polymerase Chain Reaction (qPCR)

Total RNA was extracted using TRIzol reagent (Invitrogen, Catalog #15596018) and measured using a NanoDrop spectrophotometer (DeNovix, Catalog #DS-11FX). cDNA was synthesized using the RevertAid First Strand cDNA Synthesis Kit (Thermo Scientific™, Catalog #K1622). The primers used to evaluate the expression levels of microglial signature genes in induced microglia cells and PBMCs are shown in Table 3 below. For some primer sequences, catalog numbers provided by Qiagen are provided instead.

TABLE 3

| Primer name | | Sequence (5′->3′) |
|---|---|---|
| MAFB | Forward | TCAACGACTTCGACCTGCTC |
| | Reverse | GTGTCTTCTGTTCGGTCGGG |
| CSF1R | Forward | ATTCATCAACGGCTCTGGCA |
| | Reverse | AGGACCTCAGGGTATGGGTC |
| C1QA | Forward | TCCCGGGAATTAAAGGCACC |
| | Reverse | ACCGTGTCGAAGATGACCAC |
| GPR34 | Forward | CCGCCACAAAACTTCTCAGC |
| | Reverse | CCAACCAGTCCCACGATGAA |
| HEXB | Forward | GATGTTGGCGCTGCTGACTC |
| | Reverse | GGGCTGTGGCTGATGTAGAA |

| | |
|---|---|
| GAPDH | QIAGEN, Catalog# (PPH00150F) |

In addition, the expression levels of TREM2 (PPH06065E), TDAG8 (U.S. Plant Pat. No. 2,137 H1A), ACAT2 (Bioneer, Korea, S-6042-S200-ACAT2), DHCR7 (S-6042-S200-DHCR7), and GRN genes were analyzed, and the primer sequences or information used therefor are shown in Table 4. For some primer sequences, catalog numbers provided by Qiagen are provided instead.

TABLE 4

| Primer name | | Sequence (5′->3′) |
|---|---|---|
| TREM2 | | QIAGEN, Catalog# (PPH06065E) |
| TDAG8 | | QIAGEN, Catalog# (PPH12137A) |
| ACAT2 | Forward | TGTGTCTGAGGTCATCTTTGG |
| | Reverse | CCATGCTGGAACAGAGTAGG |
| DHCR7 | Forward | GCCCAGCTCTATACCTTGTG |
| | Reverse | GCCGGGTAGAAACTTATGGC |
| GRN | Forward | CTGGACCCCGGAGGAGC |
| | Reverse | ACGGTAAAGATGCAGGAGTGG |

The above synthesized cDNA was amplified using primers and Power SYBR Green PCR Master Mix using Applied QuantStudio™ Design & Analysis Software v1.5.1 (Thermo Fisher Scientific) at 95° C. for 10 minutes, followed by 40 cycles consisting of 15 seconds at 95° C. and 1 minute at 60° C. To investigate the specificity of the amplification, a melting curve was generated. Relative quantity (RQ) levels were calculated by the 2-^{ΔΔCt} method using glyceraldehyde 3-phosphate dehydrogenase as a standard internal control group.

### Immunocytochemical Analysis

Cells obtained from the subject were fixed with 4% paraformaldehyde and permeabilized with 0.3% Triton X-100 for 10 minutes. Indirect immunofluorescence was performed using the following primary antibodies: rabbit anti-IBA1 (Wako; #019-19741; 1:400), rabbit anti-purinergic receptor P2Y12 (P2RY12; Abcam; ab140862; 1:200), rabbit anti-transmembrane protein 119 (TMEM119; Novus; NBP2-30551, 1:200), and rabbit anti-TDAG8 (Alomone labs; AGR-043; 1:1000). The cells were cultured overnight at 4° C. with primary antibodies diluted in 0.3% Triton X-100 in PBS containing 10% bovine serum albumin and 3% fetal bovine serum. After rinsing three times with PBS for 5 minutes, Alexa 488-or Alexa-594-conjugated secondary antibodies (Abcam) were used as secondary antibodies for detection. The nuclei of the cells were counterstained using 4′6-diamidino-2-phenylindole (DAPI; Sigma). Cells without primary antibody were used as a negative control group. Fluorescence images were obtained using a confocal microscope (LSM 700, Carl Zeiss, Jena, Germany).

4,4-Difluoro-1,3,5,7,8-pentamethyl-4-bora-3a,4a-diaza-s-indacene (BODIPY) Staining

To observe lipid droplet accumulation within induced microglia cells, BODIPY staining and modified Marschallinger's work were performed (Marschallinger J, Iram T, Zardeneta M, Lee S E, Lehallier B, Haney M S, et al. Lipid-droplet-accumulating microglia represent a dysfunctional and proinflammatory state in the aging brain. Nat Neurosci 2020; 23 (2): 194-208.). BV2 cells were seeded in 5×10⁴-cell 24-well plates, and induced microglia cells differentiated from 2×10⁵ PBMCs were analyzed. To generate a positive control group for BODIPY staining, BV2 cells were treated with lipopolysaccharide (LPS, 5 uM; sigma) for 18 h, fixed with 4% PFA for 10 minutes, and washed in DPBS. Induced microglia cells, LPS-treated BV2 cells, and LPS-untreated BV2 cells were cultured with BODIPY493/503 (1:1000, Thermofisher) in PBS for 10 minutes at room temperature. Cells were washed in DPBS and mounted on slides with DAPI mountant. At least three randomly selected fields were photographed using a confocal microscope (TCS SP5, Leica) (magnification x40).

### Phagocytosis Analysis

To quantify beads engulfed by phagocytosis of induced microglia cells, induced microglia cells optimally cultured for 21 days were treated with 2 μl of red fluorescent latex beads (2 μm, Sigma-Aldrich, St. Louis, MO, USA) for 2 hours at 37° C. Then, 2 ml of ice-cold PBS was added to stop the phagocytosis. The cells were washed twice with ice-cold PBS, fixed, stained with a microglial marker (IBA-1), and counterstained with DAPI. Cells were analyzed using a confocal microscope (TCS SP5-II, Leica).

### Enzyme-Linked Immunosorbent Assay (ELISA)

To quantify plasma concentrations of C-reactive protein, a C-reactive protein ELISA kit (R&D systems, #DCRP00) was used according to the manufacturer's instructions.

### RNA Sequencing and Data Analysis

Induced microglia cells from 10 normal control subjects and 14 panic disorder patients that passed raw read data quality control (QC) were selected for RNA sequencing. RNA sequencing was analyzed according to our previous study (You M J, Rim C, Kang Y J, Kwon M S. A new method of obtaining bankable and expandable adult-like microglia in mice. J Neuroinflammation 2021; 18 (1): 294.). Gene classification was based on a search performed using DAVID (http://david.abcc.ncifcrf.gov/) and Medline databases (http://www.ncbi.nlm.nih.gov/). Clustering heatmaps were generated using MeV software (https://mev.tm4.org). For Genet Set Enrichment Analysis (GSEA), the GSEA program and molecular signature database developed by the Broad Institute were used.

### Statistical Analysis

For more accurate analysis, outliers were identified and normality tests were performed. Statistical outliers were removed using the ROUT test (Q<0.5%). The normality of the cleaned data set with statistical outliers removed was tested using the Kolmogorov-Smirnov and Shapiro-Wilk tests. Unpaired and paired t-tests were used to compare microglial genes between PBMCs and iMGs to assess the statistical significance of differences between groups. Additionally, Spearman correlation analysis between panic symptom severity and gene expression was performed using GraphPad Prism version 7 for Mac (GraphPad, La Jolla, CA). Statistically significant differences are shown: **** p<0.0001, *** p<0.001, ** p<0.01, and *p<0.05.

### Measurement of State and Trait Symptoms

State symptoms were assessed using previously published PDSS; Beck Depression Inventory-II; and Beck Anxiety Inventory. Symptoms associated with PD trait vulnerability were measured using the Anxiety Sensitivity Index-Revised (ASI-R); the harm avoidance (HA) subscale from the Temperament and Character Inventory; and the neuroticism subscale from the Neuroticism-Extraversion-Openness Personality Inventory.

1. A composition for diagnosing an anxiety disorder, comprising a preparation for measuring an expression level of mRNA of an Acetyl-CoA acetyltransferase 2 (ACAT2) gene or a protein encoded by the gene.

2. The composition of claim 1, wherein the measurement of the expression level is performed in microglia of a subject.

3. The composition of claim 2, wherein the microglia are induced from peripheral blood mononuclear cells (PBMCs) isolated from the subject.

4. The composition of claim 2, wherein the microglia are prepared by treating peripheral blood mononuclear cells isolated from the subject with recombinant human granulocyte-macrophage colony-stimulating factor (GM-CSF) and recombinant human interleukin-34 (IL-34).

5. The composition of claim 1, wherein the anxiety disorder is selected from the group consisting of phobic disorder, generalized anxiety disorder, obsessive compulsive disorder, post-traumatic stress disorder, panic disorder, agoraphobia, separation anxiety disorder, and selective mutism.

6. The composition of claim 1, further comprising a preparation for measuring an expression level of mRNA of any one gene selected from the group consisting of T-cell death-associated gene-8 (TDAG8), triggering receptor expressed on myeloid cells 2 (TREM2), 7-dehydrocholesterol reductase (DHCR7), and progranulin (GRN), or a protein encoded by the gene.

7. A diagnostic kit for anxiety disorder, comprising a preparation for measuring an expression level of mRNA of an ACAT2 gene or a protein encoded by the gene.

8. The kit of claim 7, wherein the measurement of the expression level is performed in microglia of a subject.

9. The kit of claim 8, wherein the microglia are derived from peripheral blood mononuclear cells isolated from the subject.

10. The kit of claim 8, wherein the microglia are prepared by treating peripheral blood mononuclear cells isolated from the subject with recombinant human granulocyte-macrophage colony-stimulating factor (GM-CSF) and recombinant human interleukin-34 (IL-34).

11. The kit of claim 8, further comprising a preparation for measuring an expression level of mRNA of any one gene selected from the group consisting of TDAG8, TREM2, DHCR7, and GRN, or a protein encoded by the gene.

12. A method of providing information necessary for diagnosing an anxiety disorder, comprising:
(a) measuring an expression level of mRNA of an ACAT2 gene or a protein encoded by the gene from an isolated biological sample;
(b) comparing the expression level of mRNA of the ACAT2 gene or a protein encoded by the gene measured in (a) with an expression level of mRNA of the ACAT2 gene or a protein encoded by the gene in a normal control group sample; and
(c) diagnosing presence of an anxiety disorder when the expression level of mRNA of the ACAT2 gene or a protein encoded by the gene in the biological sample is higher than that in the normal control group sample.

13. The method of claim 12, wherein the biological sample is microglia.

14. The method of claim 12, wherein the microglia are induced from peripheral blood mononuclear cells.
